(19) United States
(12) Patent Application Publication
(10) Pub. No.: US 2005/0089969 A1
(43) Pub. Date: Apr. 28, 2005

(54) Insulin mimetic amino acid sequences

(75) Inventors: Josef H Wissler, Bad Nauheim (DE); Gilda Georgi, Friedrichsdorf (DE); Gunther Boehm, Echzell (DE)

Correspondence Address: BACON & THOMAS, PLLC, 625 SLATERS LANE, FOURTH FLOOR, ALEXANDRIA, VA, 22314, US

(73) Assignee: N. V. NUTRICIA, HM Zoetermeer (NL)

(21) Appl. No.: 10/476,184

(22) PCT Filed: Oct. 7, 2002
(86) PCT No.: PCT/EP02/11214

(30) Foreign Application Priority Data
Oct. 9, 2001 (DE) 101 49 668.0

(51) Int. Cl. C07H 21/04; C07K 14/62

(52) U.S. Cl. 435/69.4; 435/320.1; 435/325; 530/303; 536/23.5

(57) ABSTRACT

The invention relates to exsulins or insulin mimetic amino acid sequences with a molecular weight of up to 55,000 daltons and with 1 to 50 basic units of following Formula (1): [−] L,V,I,A]-X¹—X²-[L,V,I,A]-[D,E]-[N,Q,M]-X₃—[C,H]—X₄ [−], whereby; [L,V,I,A], [D,E], [N,Q,M], [C,H] and X^{1 to 4} represent peptidically linked amino acids; [L,V,I,A] represents leucine (L), valine (V), isoleucine (I) or alanine (A); [D,E] represents asparaginic acid (D) or glutamic acid (E); [N,Q,M] represents asparagine (N), glutamine (Q) or methionine (M); [C,H] represents cysteine (C) or histidine (H); both groups [L,V,I,A] in a basic unit can be identical or different; X¹, X², X³ and X⁴ represent any type of amino acid and the four amino acids X^{1 to 4} in a basic unit can be identical or different. The inventive exsulins can be used in dietetic and pharmaceutical agents, and to be precise, can be used for mimicking the properties and functions of members of the family of endogenous insulin and IGF proteohormones/mediators and for preventing, treating and influencing hormonal states and disturbances as well as various degenerative diseases of the body of mammals including various forms of hormonal metabolic disturbances, hormonal resistances, hormonal deficiencies, hyperinsulinemia, diabetes mellitus and autoimmune diseases as well as neurodegenerative and secondary diseases.

The invention concerns insulin-mimetic amino acid sequences, which according to the invention are also referred to as exsulins, pharmaceutical and dietetic agents containing these exsulins, the use of these exsulins as insulin-mimetics and a process for the production of these exsulins.

Insulin and IGFs are endogenous protein hormones or protein mediators that have already long been known. The many members of the known metal ion-regulated insulin proteo-hormone and mediator family are characterised by a great variety of relationships as regards structure, action and the mechanisms of biological information transfer. The best known action of the insulins themselves is their anabolic hormone action for the regulation of the glucose level in the blood. In contrast to this, the IGFs which are structurally homologous thereto are mainly known as hyperplastic growth factors with a proliferative action on various cells. Or again, inter alia the hypertrophic growth factors for nerve cells (“Nerve Growth Factors”, NGF) also belong to this family of insulin-like proteomediators, etc.

Insulin and IGFs are of major practical and socio-economic importance in a large number of medical and biotechnological use fields and as endogenous active substances as drugs for prophylaxis against and treatment of many degenerative diseases (for example various forms of diabetes mellitus and secondary diseases). Endogenously also after their synthesis from parent molecules, they are physiologically mainly stored endogenously as such in finished, functionally independent form in certain cells and thus secreted endogenously into biological fluids, whence they can display their biological activity endogenously (endocrine or paracrine and autocrine intercellular signal transfer). As an indication of the exceptionally diverse and manifold aspects of the qualitative and quantitative test systems and measurement methods and of the importance of insulin and the family of insulin-related proteohormones/mediators, for example the following selection of literature can give more detailed insights: Raizada, M. K., & LeRoith, D. (eds.): The Role of Insulin-Like Growth Factors in the Nervous System, Ann. New York Acad. Sci. 692, 1-334 (1993); Flier, J. S.: Big Deal About Little Insulin, Nature Med. 5, 614-615 (1999).

The purpose of the present invention is to provide compounds which have mimetic qualitative and functional affinities and relationships to insulin and the members of the family of insulin-like growth factors.

The compounds according to the invention are insulin-mimetic amino acid sequences or substance structures which according to the invention are also described as exsulins. The exsulins according to the invention have a molecular weight of up to 55,000 Daltons and have 1 to 50 basic units of the following general formula I:
[−] [L,V,I,A]-X¹—X²-[L,V,I,A]-[D,E]-[N,Q,M]-X³—[C,H]—X⁴ [−] (I)
wherein
[L,V,I,A], [D,E], [N,Q,M], [C,H] and X^{1 to 4} mean peptide-linked amino acids,
[L,V,I,A] stands for leucine (L), valine (V), isoleucine (I) or alanine (A),
[D,E] stands for aspartic acid (D) or glutamic acid (E),
[N,Q,M] stands for asparagine (N), glutamine (Q) or methionine (M),
[C,H] stands for cysteine (C) or histidine (H),
the two groups [L,V,I,A] in one basic unit can be the same or different, X¹, X², X³ and X⁴ mean any amino acid and the four amino acids X^{1 to 4} in one basic unit can be the same or different,
the basic unit of the general formula (I) can be located centrally or terminally,
in a terminally located basic unit only one of the residues [−] is present, if several basic units of the general formula (I) are present in one molecule, these basic units can be the same or different, and
the residues [−], if present, each stand for any amino acid peptide-linked with [L,V,I,A] or X⁴ or for an amino acid sequence built up of several of any amino acids, which is peptide-linked with [L,V,I,A] or X⁴, and
[−], if several basic units of the general formula (I) are present, stand for an indirect linkage of any amino acid or an amino acid sequence described above or for a direct linkage of two adjacent basic units. In addition to this, substance structures with insertions, deletions and conservative substitutions of amino acids can occur.

Also included according to the invention are the phosphorylated, acetylated, farnesylated, oxidised, carbohydrate and/or lipid conjugated and/or solid or liquid carrier immobilised derivatives of these exsulins.

Thus the exsulins according to the invention have 4 amino acids X in the basic unit, which can be of any nature. Thus there are no particular restrictions as regards these amino acids X^{1 to 4}. Any amino acid whatever can be present.

The basic unit of the exsulins according to the invention also has 5 amino acid groups, which are placed in square brackets in the general formula I and which can only have certain meanings.

Thus for example the amino acid groups [L,V,I,A] can only stand for leucine (L), valine (V), isoleucine (I) or alanine (A). Analogously, the amino acid groups [D,E], [N,Q,M] and [C,H] can have the meanings stated above.

Incidentally, in the context of the present invention, the amino acids are represented in the single-letter alphabet in accordance with the international nomenclature according to the state of the technology. X stands for a freely selectable amino acid or derivatisation.

The aim of writing the aforesaid amino acid groups in [brackets] is to highlight the fact that it is so-called allowed conservative substitutions or derivatisation of structural components is what is involved here. In other words, valine represents a conservative substitution of for example leucine, isoleucine or alanine, etc. The same applies analogously for the other amino acids.

The hyphen [−] shown in brackets at both ends of the basic unit of the general formula (I) highlights the fact that the basic unit of the general formula (I) can also be part of a larger molecule. Thus the hyphens [−] in themselves are not part of the basic unit of the general formula (I), but stand for other amino acid residues and/or linkages, which will be described in still further detail below.

Thus according to the invention 1 to 50 basic units or basic structures can be present in the exsulins according to the invention. By the range statement 1 to 50, all whole number values lying between the range limits are included. In order to meet the requirements for a sufficient disclosure, this means that the exsulins according to the invention can have 1,2,3,4,5,6,7,8,9,10,11,12,13,14,15,16,17,18,19,20,21,22,23,24, 25,26,27,28,29,30,31,32,33,34,35,36,37,38,39,40,41,42,43,44,45,46,47,48,49 and 50 basic units.

Should two or more basic units be present, these can be the same or different. In addition, the basic units can be located centrally or terminally. With a terminally located basic unit, this means that one of the residues or groups stated by [−] is not present. The other residue [−] then means any other amino acid sequence, which can also have one or several basic unit(s) of the general formula (I). If the basic unit is a centrally located basic unit, then both residues or groups [−] stand for any amino acid or for any amino acid sequences which can each also have one or several basic units in the chain. In the case of two adjacent or consecutive basic units, the two residues or groups [−] facing one another together stand for the amino acid sequence linking these basic units, which is built up of any peptide-linked amino acids. Here there is thus an indirect linkage of the adjacent basic units. However, the residues or groups facing one another can also mean a direct peptide linkage and hence a direct succession of two basic units.

The amino acid groups [L,V,I,A], [D,E], [N,Q,M] and [C,H] and the amino acids X¹, X², X³ and X⁴ can also be in any order in the basic unit of the general formula 1. In other words, the said amino acid groups and amino acids X¹ to X⁴ can be in any order. However, the order that can be seen from the formula (I) is preferred.

In the following description of specific exsulins, the amino acids making up the basic unit are moreover written in bold type and underlined.

The exsulins according to the invention can be obtained from organisms, tissues, cells and biological fluids, cultures and culture supernatant solutions thereof, preferably from foods and animal foods, dietary and luxury products and substitute materials and additives, in particular from milk and/or proteins, for example caseins. If for example caseins are used as starting materials, then in the context of the present documents the exsulins according to the invention are also described as casoinsulins.

Thus the exsulins according to the invention are not present in the staring materials as functionally independent substance structures, but they are only cryptically structurally and functionally present, i.e. they are only formed in subsequent reactions and processes and/or derived from independent parent molecules, which have no functional properties and functional affinities in the meaning of the invention.

Thus, the exsulins according to the invention are preferably obtainable from the starting materials described.

The exsulins according to the invention have metal-regulated properties and interactions with other substances, which are effected in particular by transition metal ions, for example Cu and Zn ions.

The exsulins according to the invention have mimetic qualitative and functional affinities and relationships to insulins and members of the family of insulin-like growth factors (“IGF”). They are however structurally different from the family of the endogenous insulin and insulin-related (IGF) proteohormones and proteomediators.

It is assumed that the properties of the exsulins according to the invention are determined by the basic unit of the general formula (I) described above, without however being bound to this explanation.

Preferred exsulins according to the invention here are as follows:

LTDLENLHLPLPLLQPSMQQVPQPIPQTLALPPQPLWSVPEPK
and
YPVQPFTESQSLTLTDVENLHLPPLLLQSWMHQPHQPLPPTVMFPPQSV
LSLSQSK

According to the invention, the exsulins or substance structures obtainable by insertions, deletions and/or conservative substitutions of amino acids are also included.

The exsulins according to the invention have the following effects and properties:

A) Selective Effects:
Mimetic qualitative and functional affinities and relationships to insulin and to members of the family of the insulin-like growth factors (“IGF”).
Insulin-mimetic effects by interaction with various cells, cell systems and organs of vertebrates, including the mammals and man.
Insulin-mimetic effects by selective interaction with insulin-producing (Langerhans islet) β-cells.
Insulin-mimetic effects by selective interaction and complex formation in vivo and in vitro with antibodies of vertebrates, including the mammals and man, to insulin.
LD₅₀ not determinable, since no lethal effects.
No endotoxin-like or similar effects.
No lytic effects on erythrocytes and leukocytes in vitro.

B) Physical-Chemical and Chemical Properties:
Typical properties of substances regulated by metals, in particular by transition metal ions, for example by Cu or Zn ions.
They interact with other metal-regulated substances with the formation of homo- and/or heterocomplexes.
They may, but do not have to, have protein or peptide properties.
They have hydrophobic, ionic and metal ion complex-binding functional groups.
They are soluble in aqueous media including 20% ethanol at a pH value of at least 4.0 to 10.0.
They are soluble in an aqueous ammonium sulphate solution of saturation 20% (0.8 moles/l) at a pH value of at least 4.0 to 10.0.
They are insoluble in chloroform, carbon tetrachloride and similar apolar, non-aqueous media.
They display abnormal hydrodynamic behaviour, so that the hydrodynamic equivalent of the molecular weight appears smaller than the true molecular weight, as far as determinable by gel chromatography, electrophoresis and membrane permeation methods.
They adsorb reversibly in structure and biological activity on anion exchangers, hydrophobic gels, calcium phosphate gel and hydroxylapatite and can without modification be subjected to volume distribution chromatography.

Because of their properties, the exsulins according to the invention can be used for mimesis of the properties and functions of members of the family of the endogenous insulin and IGF proteohormones/mediators (as described above) and also as substitutes for these.

Also an object of the invention are pharmaceutical and dietetic agents, which contain at least one exsulin according to the invention. These agents can in particular be used for prophylaxis against and for the specific treatment or influencing of hormonal states and disorders and of various degenerative diseases, for example of various forms of hormone metabolism disorders, resistances, deficiencies, hyperinsulinaemia, diabetes mellitus and autoimmune and neurodegenerative diseases and diseases secondary to these.

A further use possibility is the production of substances inhibiting and regulating the effects of the members of the family of the endogenous insulin and IGF proteohormone/mediators and of molecular biological equivalent structures and of antibodies.

The exsulins according to the invention can be administered alone or as a mixture in the form of normal drugs and in accordance with normal safety, testing and regulatory guidelines systemically or locally, parenterally or intravenously, in mammals in an amount of 1 fg/kg to 10 g/kg. To meet the purposes, these drugs can also contain at least one anti-exsulin immunoglobulin and/or molecular biological equivalent structure. Correspondingly, the substances of the invention can be used as dietetic agents or additives.

As well as at least one exsulin according to the invention, the drugs according to the invention can also contain at least one other carrier, auxiliary or additive substance. In addition, one further or several further active substance(s) can be present.

The exsulins according to the invention can also be incorporated in dietetic agents. This includes foods, dietary agents, food supplements and luxury goods and substitute and additive substances.

The exsulins according to the invention can be produced or obtained/extracted by treating the whole or parts of organisms, tissues, cells and biological fluids, cultures and culture supernatants thereof, preferably of foods and animal foods, dietary and luxury goods and substitute and additive substances, in particular from milk and/or milk proteins, for example caseins, with physical, chemical and/or biological processes in vivo and/or in vitro in such a manner that parts thereof are removed, added, selected and/or modified, for example in order to procure maternal milk-like properties for the agent, or to remove such properties from the agent. In addition, food and animal food technology, medical technology, molecular biotechnology, membrane technology, pharmaceutical technology, cell biology, cell culture technology, immunobiotechnology, enzyme technology, chromatography and known conventional processes can be used alone or in combination, which can be optimised for manageability, agent quality, quantity, yield, economy and other usual norms and standards. If required and permitted (for example taking account of ethical principles and/or the foods, medicines and other laws and regulations), for the production and extraction of the substances and agents, it is also possible to start from known structures of food, animal food, dietary and luxury goods and also substitute and additive substances and to use these, and also from chemically and/or molecular biologically synthesised sequences of molecule components and/or from parts and homologous sequences thereof.

For this, in particular animals, plants, fungi, seeds, microorganisms, hybridomas, gene biological, gene therapeutic and transgenic recombinants thereof and organisms derived therefrom, organs, tissues, cells, biological fluids, exudates, eggs, blood, lymphs, milk, wheat, oats, beans, algae as a whole or parts thereof or in combination in their own phases as such, extracts thereof, or those immobilised on boundary areas alone, mixed, with and without additives as such, homogenised alone or in combination can be processed and used.

In principle, the modifications of the starting substances which lead to the exsulins according to the invention with the described properties can be effected by various processes of material and energy input. For example, for the chemical cleavage of parent molecules, or the removal of functional groups, the energy input can take place by heating and/or by pH changes. Nowadays, however, enzyme technology processes are preferably used for this. In particular, hydrolases (E.C. 3.-.-.-), for example proteases (E.C. 3.1.4.-) and phosphatases (E.C.3.1.3.-) can be used as biocatalysts alone or in combination with other agents. It has for example been found especially advantageous when trypsin (E.C. 3.4.21.4) and/or chymotrypsin (E.C. 3.4.21.1) are used each alone or in combination as proteases and an acid phosphatase (E.C.3.1.3.2) as phosphatase and here especially preferably the last-named enzyme from potatoes is used.

Plant and fungal organisms, parts, derivatives and food and animal food industry products produced therefrom can also be modified by material or by energy input in such a manner that exsulins according to the invention are formed. Plant-derived phosphate-containing substances, plant-derived highly phosphorylated carbohydrates and phosphate-containing proteins can also preferably be treated with phytases (E.C. 3.1.3.-, for example inositol hexakisphosphate phosphohydrolases, E.C. 3.1.3.8) alone or in combination with phosphatases. In these cases, the products obtained in particular are more advantageously further treated in such a manner that the free phosphate formed as a result therein is removed from the product by suitable processes before it is used.

According to a preferred process for the production and extraction of the exsulins according to the invention, milk, parts and derivatives thereof and food and animal food industry products and dietary or luxury agents, substitute and additive substances produced therefrom are modified by enzyme technology in such a manner that the exsulins according to the invention are formed. In an especially preferred process, cows' milk is used as milk, or the casein and/or cleavage products thereof (casopeptides) are used as part of the cows' milk. In addition, substance components in the form of the phosphoproteins and phosphorylated cleavage products therefrom (“Phosphopeptides”), e.g. those of bovine casein and cleavage peptides thereof, can also further be treated with the said, especially preferred phosphatase form for the dephosphorylation. For example, the casein of cows' milk or its proteolytic cleavage products can be taken up in neutralised water and divided into two batch portions; one of these is subjected to the said treatment for the dephosphorylation, in particular with the especially preferred phosphatase form. The preparation which was subjected to the dephosphorylation reaction, contains the compounds according to the invention freed from phosphate groups, or depleted in them; the other, effectively untreated preparation contains the exsulins according to the invention as naturally phosphorylated compounds.

According to an especially preferred process, food, animal food, dietary or luxury products, substitute and additive materials with an epidemiologically substantiated risk of the development and occurrence of degenerative diseases in connection with nutritional factors, nutritional forms and diets are modified in such a manner that the exsulins according to the invention are removed therefrom in cryptic or independent form, i.e. before or after formation or production of the substances according to the invention. In principle, these changes or the selection of means which lead to the desired properties, can be effected by various procedures for the substance and energy input. For example, for this the energy input can be effected by heating and/or pH changes, followed by chemical, physical-chemical and/or physical or mechanical separation procedures. Preferably, however, adsorption and complexing processes in the meaning of the already described processes are used for this.

The individual exsulins according to the invention can also be produced as such in isolated form. For this, the exsulins formed (casoinsulins) are separated from other substances and isolated and/or concentrated up and/or converted into a liquid (dissolved) or solid state by normal enrichment, purification and isolation processes (“downstream processing”) by physical, physical-chemical, chemical and/or (also gene and/or immuno-) biological methods in accordance with the state of the technology. Preferably, the exsulin-(casoinsulin)-containing solutions are concentrated to a dry mass content of 35-40% in accordance with the state of the technology.

These downstream processing operations can be configured as single-step or multistep batch-(one pot) and/or column processes alone or combined with other processes. For this, firstly normal processes for the preparation of biological substances can be used, preferably chromatography, electrophoresis membrane technology and sedimentation processes, for example precipitation processes, and phase extraction processes. For example, liquid phase extraction processes in the form of countercurrent distribution, in particular in the form of the thin film countercurrent distribution usual according to the state of the technology, can advantageously be used. As chromatographic processes, in particular ion exchanger, gel, zone precipitation, affinity, hydrophobic chromatography and/or filtration are used. In the latter, immunobiological, hybridising or complexing compounds or those with metal ion affinity and other affinities bound to solid, liquid and/or gas phases are used. Solid phases can for example be membranes, gels, hydroxylapatite, ceramics, glass particles, composite materials and/or combinations thereof. They can also be used unbound, for example in so-called hybrid and/or hollow fibre module processes. The concentration is effected by normal methods, for example by precipitation, countercurrent distribution, complex formation, membrane permeation processes, in particular dialysis and/or ultrafiltration on suitable membranes, evaporation and/or drying, preferably spray drying, for example in the form of lyophilisation.

For the preparation of the isolated exsulins according to the invention, both batches are then transferred into a medium not harmful to the exsulins; preferably as such a medium an aqueous liquid with physiological, neutral conditions and component concentrations in the range from 1 fmole/l to 5 moles/l is transferred; a specific example thereof is a physiological, aqueous medium of 0.15 mole/l NaCl, controlled pH range 6.8-7.4 and a buffer of 1 mmole/l imidazole HCl, wherein all reaction partners are adjusted to reaction equivalence in the concentration range from 1 to 20 mmoles/l. In one batch, (containing for example dephosphorylated cleavage products of the bovine caseins) copper ions are added for the isolation of the exsulins according to the invention. In another batch (containing for example phosphorylated cleavage products of the bovine caseins) zinc ions are added for the isolation of the exsulins according to the invention. After a reaction time of 0.01 to 100 hours at 1-60° C., preferably 0.1 to 10 hours at 10-40° C., for example at least half an hour and normal ambient conditions, both batches are worked up separately.

With the use of transition metal ions, for example Cu and/or Zn ions, the preparation with the exsulins according to the invention contains these as copper or zinc complexes. The copper complexes are recognisable in concentrated form and at normal temperature in a bluish colour. The colourless zinc complexes are partly present in dissolved colourless and in insoluble, white form in the preparation and are in mutual equilibrium in the solubility product equilibrium. The insoluble form can be separated from the soluble form after centrifugation under normal conditions (10,000×g) as a precipitate and used separately after a washing procedure with water or buffer. From both preparations of the metal ion-complexed substances according to the invention, various UV spectra and hence also a concentration measurement, on the dry weight basis in absolute concentration can be obtained by means of the extinction coefficients in accordance with the Lambert-Beer law.

The workup of the batches is preferably performed with the use of membrane technology for the dialysis or ultrafiltration. For this, membranes are used which are characterised and tested by a so-called exclusion limit of the hydrodynamic equivalent of the molecular mass (“molecular weight”) of 100 to 30,000 Daltons. In a specific example membranes which have such an exclusion limit of 500 Daltons are preferably used.

Examples of such membranes, their handling, processing, preparation, handling for sterile operations and freedom from pyrogens, and equipment for this, etc., are known to the skilled person in accordance with the state of the technology from the normal literature; on this, see: J. H. Wissler: Large scale and biotechniques for the production and isolation of leucocytic effector substances of regenerative tissue morphogenesis by culturing cells in serum-free, synthetic fluids: Design, preparation and use of a novel medium. Cox, P. H., Ed. Developments in Nuclear Medicine Series, Vol 7: Fueger, G. F., Ed. Blood Cells in Nuclear Medicine, Part 2: Migratory Blood Cells. Martinus Nijhoff Publishers, Boston, 1984, p. 393-471. According to an especially preferred process, the ultrafiltration is performed under the stated, especially preferred conditions and the buffer used for this for example until exchange of the medium in the volume ratio of at least 1:1000 (batch volume:ultrafiltrate) has taken place and hence all non-reacting components have been removed with the ultrafiltrate. Both batches are collected separately and can be sterilised in the usual way by a filtration on a membrane of 0.01-5 μm, in particular for example 0.2 μm pore width and kept stable, for example frozen, until further use.

Between the production and isolation processes presented above, the solutions of the biologically active substances of the invention obtained can be concentrated for subsequent processes or separation of other substances, for example also salts. This concentration (separation of a major part of the solvent medium) can be effected in various ways. For example, for this the substances of the invention can be processed by lyophilisation and/or ultrafiltration or dewatering dialysis on one of the membranes described, in particular one with an, exclusion limit of 500 Daltons.

For therapeutic and dietetic use, the exsulins (casoinsulins) are preferably isolated by at least on of the stated steps. Preferred is an embodiment which frees the substances of the invention from the major part of the contaminants through a combination of at least two of the stated steps.

Further, the exsulins according to the invention can also be produced synthetically or “artificially” by the currently known methods for the chemical synthesis of peptides.

The temperature and pH conditions are not particularly critical during the performance of the isolation, production, use and storage steps. If it is intended to obtain the natural, biologically active form of the substances/exsulins according to the invention, it is advisable to maintain a temperature in the range from about −80 to 70° C., in particular 0 to 40° C., preferably 4-20° C. Further, the separation and purification steps must be performed under essentially physiological pH and salt conditions. A significant advantage of the process according to the invention consists in that the maintenance of these conditions is readily possible. If necessary, the substance solution can be treated with antioxidants for the prevention of oxidation effects, for example adapted to or in accordance with the physical states and use purposes of the substances of the invention, with inosenols, L-ascorbic acid (Vitamin C) or L-cysteine. In the storage and use of a non-frozen solution of the exsulins according to the invention between 0 to 50° C., it can be advantageous to add additives to the solution which do not damage the exsulins according to the invention, and also hold them in solution, and those which hinder or prevent the growth of possible microbial contaminants. Examples of such additives are 0.5 to 3 mole/l NaCl, salting ammonium sulphate, NaN₃, organic solvents (for example additions of ethanol), and antibiotics.

The exsulins (casoinsulins) according to the invention can be kept, stored and used in a medium not damaging these substances. As such a medium, preferably water or an aqueous liquid, also one complemented with salts and/or cell culture media is used, during which a controlled pH range of 3-11, in particular 5-9 is established. A specific example of this with physiological, neutral conditions is a salt solution of 0.15 mol/l NaCl and a 1 mmole/l buffer, for example phosphate or imidazole, pH 6.8-7.4. The exsulins according to the invention can be kept unmodified and biologically active at room temperature, preferably frozen (at ≦−25° C.) after normal sterilisation, for example methods of filtration and filters with a pore width of 0.2 μm.

For all process steps, water of the ASTM-1 quality according to the state of the technology is used; see. ASTM D-1193-70: Standard Specification for Reagent Water, Annual Book of ASTM Standards, Easton Md., ASTM 1970. Furthermore, it was filter-sterilised on surfactant-free membranes of 0.2 μm pore size and freed from possible endotoxin contamination by ultrafiltration on surfactant-free membranes with an exclusion limit of 1,000 Daltons (sterile, pyrogen-free water of ASTM-1 quality); on this, see the aforementioned publication by J. H. Wissler: Large scale and biotechniques for the production and isolation of leucocytic effector substances of regenerative tissue morphogenesis by culturing cells in serum-free, synthetic fluids: Design, preparation and use of a novel medium.

The invention is explained in more detail below on the basis of examples describing preferred embodiments.

### EXAMPLE 1

Production of Insulin-Mimetic Casopeptides (Casoinsulins) as Supplement for Foods or as Supplement in Capsule or Sachet Form:

Caseinate, obtained from bovine milk, is dissolved at a concentration of 10% in warm water at 60° C. and the solution is pasteurised. After cooling of the solution to 40° C. the pH value is adjusted to 7.0 with diluted sodium hydroxide solution. Trypsin is then added (enzyme:substrate ratio of 1:250) and the solution incubated for 120 minutes at 40° C. Next, the same quantity of chymotrypsin is added and the solution incubated at the same temperature for a further 30 mins. The pH value is checked at intervals and if necessary again adjusted to 7.0. After completion of the hydrolysis, the solution is kept at 85-90° C. for 10 mins to inactivate the enzymes. The casoinsulins are isolated by affinity ultrafiltration or affinity chromatography or after complexing with zinc chloride (2-5 mmoles/l) (Example 7 and 8) and then dried (spray or freeze-drying). In this form, the casoinsulins can be added to other foods as supplements or be administered in capsule or sachet form.

### EXAMPLE 2

Production of a Drink or Special Probe Food for Patients with Diabetes Type II [NIDDM] on the Basis of a Casein Hydrolysate:

120 kg of casein (90% protein) are dissolved in warm water at 60° C. After a pasteurisation step, the batch is cooled to 40° C. and a mixture consisting of the proteases trypsin and chymotrypsin in the ratio 1:1 is added (enzyme:substrate ratio of 1:250). The batch is incubated for 3 hours at 40° C. After this, a further 100 g to 5 kg of the casoinsulins produced in Example 1, 265 kg of carbohydrates (fructose and starch) and 100 kg fat (animal and plant), minerals, trace elements and vitamins are added. After the complete dissolution of all components, the solution is homogenised and finally sterilised.

### EXAMPLE 3

Production of a Infant Follow on Formula with Tryptically Hydrolysed Dephosphorylated Casein:

140 kg of casein, which had been 60% dephosphorylated with acid phosphatase (90% protein), are dissolved in warm water at 60° C. After a pasteurisation step, the mixture is cooled to 40° C. and trypsin is added (enzyme:substrate ratio of 1:300). The solution is incubated at 40° C. for 2.5 hrs. After inactivation of the enzyme at 85-90° C. for 10 minutes, a two-stage ultrafiltration is performed. 1^{st} stage: ultrafiltration of the hydrolysate solution with a separation limit of 50,000 Daltons (Da); 2^{nd} stage: ultrafiltration of the permeate from the first stage with a separation limit of 1000-3000 Da. To the casoinsulins now concentrated in the retained liquid are consecutively added 290 kg of powdered whey (13% protein), 67 kg of whey protein concentrate (76% protein), 154 kg of lactose, 49 kg of maltodextrins, 285 kg of a suitable lipid mixture and the quantities of minerals, trace elements and vitamins recommended for baby foods. After the complete dissolution of all components, the solution is homogenised, pasteurised and evaporated to a dry mass content of 35-45%. As a final step, a spray drying is performed.

### EXAMPLE 4

Production of Insulin-Mimetic Peptides from a Mixture of Soya and Wheat Proteins as a Supplement for Foods or as a Supplement in Capsule or Sachet Form:

Soya and wheat proteins are mixed in a 60 to 40 ratio. Next, this mixture is dissolved at a protein concentration of 6-10% in warm water at 45° C. and the solution is pasteurised. After cooling of the solution to 40° C., the pH value is adjusted to 7.0 with dilute sodium hydroxide solution and a mixture of trypsin and chymotrypsin (1:1) with an enzyme:substrate ratio of 1:150 is added and the solution incubated at 40° C. for 150 mins. The pH value is checked at intervals of ca. 20 min and if necessary again adjusted to 7.0. After completion of the hydrolysis, the solution is heated at 85-90° C. for 10 minutes to inactivate the enzymes. The insulin-mimetic peptides are concentrated or isolated in the form of zinc complexes by ultrafiltration (see Example 7 and 8) and used in the sense of the invention as a mixture together with other substances or as isolated substances, e.g. in capsule or sachet form.

### EXAMPLE 5

Production of a Dietary Supplement Material for the Therapeutic Support of Patients with Diabetes Type I on the Basis of a Glycomacropeptide (GMP) Hydrolysate:

100 kg of glycomacropeptide (isolated from bovine sweet whey proteins) consisting of 75-100% GMP and 0-25% whey proteins are dissolved in warm water at 60° C. (5-15% protein solution). After a pasteurisation step, the batch is cooled to 40° C. and trypsin added in an enzyme:substrate ratio of 1:150. The batch is incubated at 40° C. for at least 2 hours. After concentration of the insulin-mimetic peptides by affinity ultrafiltration or after complexing with zinc salts (Example 7 and 8), these are dried (spray or freeze-drying) and can be used thus in sachet, tablet or capsule form.

### EXAMPLE 6

Production of a Infant Follow on Formula with the Use of Insulin-Mimetic Peptides Obtained from Bovine Whey Proteins:

60 kg of whey protein concentrate (76% protein) are dissolved in warm water at 55° C. After a pasteurisation step, the batch is cooled to 40° C. and trypsin added in an enzyme:substrate ratio of 1:250. The mixture is incubated at 40° C. for 2.5 hours. After an initial ultrafiltration of the hydrolysate solution with a separation limit of 50,000 Da, the permeate is concentrated in a second step with a separation limit of 1000-3000 Da. The insulin-mimetic peptides contained in the retained liquid are then isolated by affinity chromatography in a batch process. The insulin-mimetic peptides are then added to a 10% casein solution in a protein ratio of 1:2 to 1:20. The remaining components such as whey powder or whey protein concentrate, lactose, lipids, vitamins, minerals and [?] are admixed in the quantities recommended for baby foods. After the complete dissolution of all components, the batch is homogenised, pasteurised and evaporated to a dry mass content of 35-45%. As a final step, a spray drying according to Example 1 is effected.

### EXAMPLE 7

Production of Isolated Substances According to the Invention and Concentration of Substances According to the Invention by Complexation Methods:

For this, the batches are transferred into a solution of 0.15 mole/l NaCl and 1 mmole/l imidazole HCl buffer with controlled pH 7.0 and adjusted to a peptide concentration of 2-5 mmoles/l. For dephosphorylated cleavage products such as for example the human and the dephosphorylated, preferably phosphatase-treated bovine caseins and casopeptides, a quantity of 2-5 mmoles/l CuCl₂ equivalent to the peptide concentration is added with monitoring of the pH value (7.0) to a batch for the isolation of the substance components. For phosphopeptides, for example the natural non-pretreated bovine caseins, in another batch for the isolation of the substance components an analogous procedure is used with addition of 2-5 mmoles/l ZnCl2. After a reaction time of at least half an hour and under normal ambient conditions, both batches are separately ultrafiltered against water (if required, against a buffered salt solution) on a membrane with the exclusion limit of the hydrodynamic equivalent of the molecular mass (“molecular weight”) of 500 Dalton, until exchange of the medium in the volume ratio of at least 1:1000 (batch volume:ultrafiltrate) has taken place. Both batches are again concentrated by ultrafiltration by adjustment to the original batch volume, collected separately, sterilised in the usual way via a filtration of a membrane of 0.2 μm pore width and if necessary frozen until further use. The dissolved copper complexes of the components are visible in concentrated form as a bluish solution at normal temperature. The colourless zinc complexes are in solubility equilibrium between insoluble (sparingly soluble) and soluble fractions. The insoluble fraction of the zinc compounds of the components can be obtained separate from the dissolved fraction, or the fraction of the components still present in solution, as a white precipitate by centrifugation at 10,000×g. A washing process with water of buffer completes the separation of soluble and insoluble fraction. From both preparations of the components, the concentrations of the substances are determined by measurement of the UV spectra in accordance with the Lambert-Beer law. The dissolved substances can be obtained salt-free by ultrafiltration with water under the same boundary conditions and as dry substance for further use after lyophilisation.

### EXAMPLE 8

Transition Metal Ion-Free Exsulins (Casoinsulins):

The neutral to weak acetic acid (pH 5.0) solution of the transition metal ion complexes obtained according to Example 7 are freed from transition metal ions by extraction (countercurrent distribution) with a freshly prepared solution of at least 100 mg/l dithizone in chloroform (green). The reaction with transition metal ions Zn++ or Cu++ ions colours the solution red to brown-violet. After phase separation, the aqueous (upper) phase is removed and the extractions (countercurrent distribution cycles) repeated (about 5 times) until the reagent chloroform phase remains green, i.e. is free from transition metal ions. The aqueous phase can be concentrated and purified (for the removal of traces of the reagents and for the separation of components) in the usual way according to the state of the technology. The substances of the invention can then be supplied to the relevant applications.

1. Exsulins or insulin-mimetic amino acid sequences with a molecular weight of up to 55,000 Daltons and with 1 to 50 basic units of the following general formula I
[−] [L,V,I,A]-X¹—X²-[L,V,I,A]-[D,E]-[N,Q,M]-X³—[C,H]—X⁴ [−] (I)
wherein
[L,V,I,A], [D,E], [N,Q,M], [C,H] and X^{1 to 4} mean peptide-linked amino acids,
[L,V,I,A] stands for leucine (L), valine (V), isoleucine (I) or alanine (A)
[D,E] stands for aspartic acid (D) or glutamic acid (E),
[N,Q,M] stands for asparagine (N), glutamine (Q) or methionine (M),
[C,H] stands for cysteine (C) or histidine (H),
the two groups [L,V,I,A] in one basic unit can be the same or different,
X¹, X², X³ and X⁴ mean any amino acid and the four amino acids
X^{1 to 4} in one basic unit can be the same or different,
the basic unit of the general formula (I) can be centrally located or terminally located, in a terminally located basic unit only one of the residues [−] is present, if several basic units of the general formula (I) are present in one molecule, these basic units can be the same or different, and
the residues [−], if present, each stand for any amino acid peptide-linked with [L,V,I,A] or X⁴ or for an amino acid sequence built up of several of any amino acids, which is peptide-linked with [L,V,I,A] or X⁴, and
[−], if several basic units of the general formula (I) are present, stands for an indirect linkage of any amino acid or of an amino acid sequence described above or for a direct linkage of two adjacent basic units, and the amino acid sequences obtained by insertions, deletions and conservative substitutions of amino acids and the phosphorylated, acetylated, farnesylated, oxidised, carbohydrate and/or lipid conjugated and/or solid or liquid carrier immobilised derivatives thereof.

2. Exsulins according to claim 1, characterized in that the basic units of the general formula (I) are as follows:
[−] LTDLENLHL [−] and [−] LTDVENLHL [−].

3. Exsulins according to claim 2, characterised in that they correspond to the following formula:
LTDLENLHLPLPLLQPSMQQVPQPIPQTLALPPQPLWSVPEPK
or
YPVQPFTESQSLTLTDVENLHLPPLLLQSWMHQPHQPLPPTVMFPPQSV
LSLSQSK.

4. Exsulins according to claim 1, characterised in that they are obtainable from the following starting materials or parts thereof: organisms, tissues, cells and biological fluids and exudates, cultures and culture supernatant solutions thereof, foods and animal foods, dietetic and luxury products and substitute and additive substances, milk and/or milk proteins.

5. Exsulins according to claim 4, characterised in that they are obtainable from caseins.

6. Dietetic or pharmaceutical agent containing at least one exsulin according to claim 1 and optionally at least one normal auxiliary, carrier or additive substance.

7. Dietetic or pharmaceutical agent according to claim 6 for mimesis of the properties and functions and substitute agents for members of the family of the endogenous insulin and IGF proteohormones/mediators and for prophylaxis against and for the treatment and influencing of hormonal states and disorders and of various degenerative diseases of the body in mammals, including various forms of hormone metabolism disorders, resistances, deficiencies, hyperinsulinaemia, diabetes mellitus and autoimmune and neuro-degenerative and secondary diseases.

8. Exsulins according to claim 1 for mimesis of the properties and functions and substitute agents for members of the family of the endogenous insulin and IGF proteohormones/mediators and for prophylaxis against and for the treatment and influencing of hormonal states and disorders and of various degenerative diseases of the body in mammals, including various forms of hormone metabolism disorders, resistances, deficiencies, hyperinsulinaemia, diabetes mellitus und autoimmune and neuro-degenerative und secondary diseases and for the production of corresponding dietetic and pharmaceutical agents and for the production of inhibitors and regulators of the effects of the members of the family of the endogenous insulin and IGF proteohormones/-mediators and of molecular biological equivalent structures thereof.


SEQUENCE LISTING (5 sequences)

SEQ ID NO: 1 - protein, 43 aa, Unknown
  mammalian
LTDLENLHLPLPLLQPSMQQVPQPIPQTLALPPQPLWSVPEPK

SEQ ID NO: 2 - protein, 56 aa, Unknown
  mammalian
YPVQPFTESQSLTLTDVENLHLPPLLLQSWMHQPHQPLPPTVMFPPQSVLSLSQSK

SEQ ID NO: 3 - protein, 11 aa, Unknown
  mammalian
XLTDLENLHLX

SEQ ID NO: 4 - protein, 11 aa, Unknown
  mammalian
XLTDVENLHLX

SEQ ID NO: 5 - protein, 11 aa, Unknown
  mammalian
XLXXLENXHXX